(19) United States
(12) Patent Application Publication
(10) Pub. No.: US 2011/0009898 A1
(43) Pub. Date: Jan. 13, 2011

(54) Pressure Bandage

(76) Inventors: Wei-Hua Lu, Neipu Hsiang (TW); Yung-Chuan Chen, Neipu Hsiang (TW); Li-Wen Chen, Neipu Hsiang (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/823,173

(22) Filed: Jun. 25, 2010

(30) Foreign Application Priority Data
Jul. 13, 2009 (TW) 098123645

(51) Int. Cl. A61B 17/00

(52) U.S. Cl. 606/202

(57) ABSTRACT

A pressure bandage comprises a banding portion, a container received a fluid and a fixing member. The container has a first and a second connecting portions, and the first and second connecting portions are respectively protruded from the opposite sides of the banding portion. At least one check valve is mounted in at least one of the first and second connecting portions for delivering the fluid into the container. The fixing member is adapted to fix the banding portion to a treatment part.

### BACKGROUND OF THE INVENTION

1. Field of the Invention

The present invention generally relates to a pressure bandage and, more particularly, to a pressure bandage that allows free movement of the user using the pressure bandage.

2. Description of the Related Art

Conventional pressurized treatment includes a negative pressure therapy for healing the open wound of the hypodermis tissue, and a positive pressure therapy for healing the wound of the epithelium tissue.

Specifically, the negative pressure therapy is to reduce the pressure of a treatment part of a user by a pump for facilitating the open wound healing.

On the other hand, the positive pressure therapy includes use of a banding portion fixed to the treatment part of the user requiring pressurized treatment. A pump delivers a fluid in a conduit such that the pressure at the banding portion is greater than the atmospheric pressure, maintaining the treatment part in a pressurized status to inhibit growth of the tissue at the treatment part.

In the conventional pressurized treatment, the user can not move freely due to limitation of the position of the pump for delivering the fluid to the treatment part via the conduit for maintaining the pressure at the treatment part. Thus, the banding portion must be removed when the user is intended to move beyond the range of the conduit due to limitation of the length of the conduit and/or the position of the pump. As a result, the treatment part of the user can not be maintained in the pressurized status, which is inconvenient to use.

### SUMMARY OF THE INVENTION

An objective of the present invention is to provide a pressure bandage allowing free movement of the user using the pressure bandage.

Another objective of the present invention is to provide a pressure bandage allowing connecting with a pump when the difference between the positive pressure in the pressure bandage and the atmospheric pressure becomes small, maintaining the pressure bandage in a positive pressure status.

The invention discloses a pressure bandage comprising a banding portion, a container received a fluid and a fixing member. The container has a first and a second connecting portions, and the first and second connecting portions are respectively protruded from the opposite sides of the banding portion. At least one check valve is mounted in at least one of the first and second connecting portions for delivering the fluid into the container. The fixing member is adapted to fix the banding portion to a treatment part.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 shows a cross sectional view of a pressure bandage according to a first embodiment of the invention.

FIG. 2 shows a top cross sectional view of the pressure bandage of FIG. 1.

FIG. 3 shows a side cross sectional view of the pressure bandage according to the first embodiment of the invention.

FIG. 4 shows a side cross sectional view similar to FIG. 3, illustrating connecting of a pump and maintaining of a positive pressure status.

FIG. 5 shows a side cross sectional view of the pressure bandage of FIG. 1 placed on a treatment part.

FIG. 6 shows a top cross sectional view of the pressure bandage according to a second embodiment of the invention.

FIG. 7 shows a side cross sectional view of the pressure bandage according to the second embodiment of the invention.

FIG. 8 shows a side cross sectional view similar to FIG. 7, illustrating connecting of the pump and maintaining of a positive pressure status.

In the various figures of the drawings, the same numerals designate the same or similar parts. Furthermore, when the term “first”, “second”, “positive”, “negative” “outer”, “bottom” and similar terms are used hereinafter, it should be understood that these terms are reference only to the structure shown in the drawings as it would appear to a person viewing the drawings and are utilized only to facilitate describing the invention.

### DETAILED DESCRIPTION OF THE INVENTION

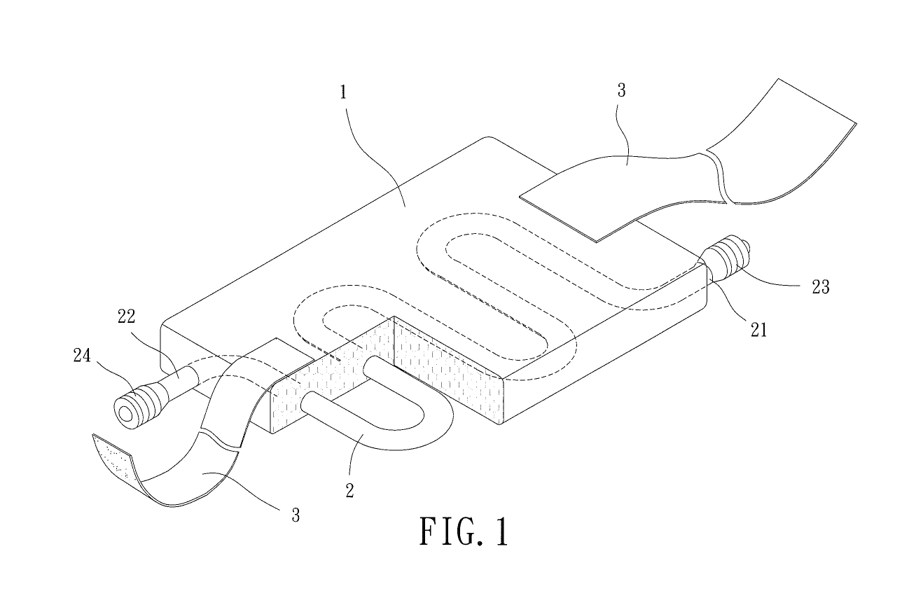

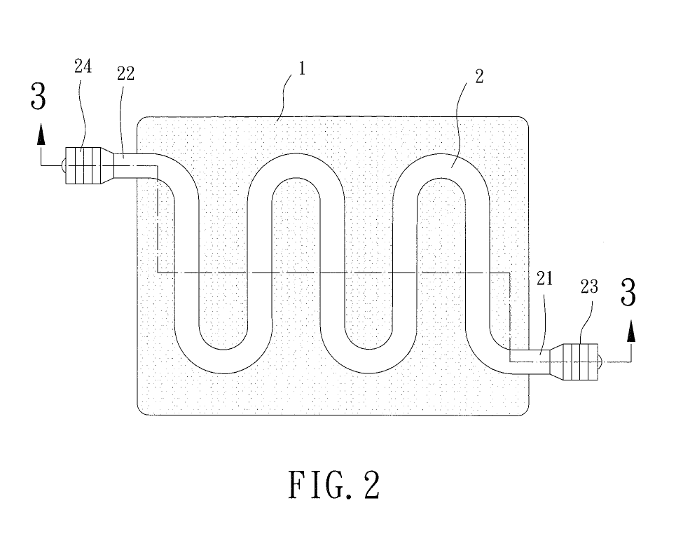

Referring to FIGS. 1, 2 and 5, a pressure bandage is disclosed according to a first embodiment of the invention. The pressure bandage comprises a banding portion 1, a container 2 received in the banding portion 1, and a fixing member 3 adapted to fix the banding portion 1 and the container 2 on a treatment part 92 of a body portion 94 of a user requiring pressurized treatment.

In the preferred form shown, the banding portion 1 is made of shapeable sponge, elastic bandage, or the like. The banding portion 1 can be of any desired shape and size.

The container 2 can be a conduit or a bladder for receiving a fluid. In the first embodiment shown, the container 2 is in the form of a conduit made of polypropylene, polyethylene or rubber, etc. The container 2 is fixed in the banding portion 1 and adjacent to a contact surface of the banding portion 1. In the preferred form shown, the contact surface is the bottom surface of the banding portion 1. The container 2 includes a first connecting portion 21 and a second connecting portion 22 in communication with the first connecting portion 21. The first and second connecting portions 21 and 22 are respectively protruded from the opposite sides of the banding portion 1. At least one check valve is mounted in at least one of the first and second connecting portions 21 and 22 for delivering a fluid into the container 2, maintaining the container 2 in a positive pressure status.

In the preferred form shown, two check valves 23 and 24 are mounted in the first and second connecting portions 21 and 22 respectively, wherein the check valve 23 mounted in the first connecting portion 21 serves as an inlet for delivering the fluid into the container 2, another check valve 24 mounted in the second connecting portion 22 serves as an outlet for delivering the fluid out of the container 2. Therefore, the fluid can circulate in the container 2, and the flow rate of the fluid can be controlled by the two check valves 23 and 24 so as to impart a positive pressure to the treatment part 92 of the user.

The fixing member 3 includes two straps respectively having a hook fastener and a loop fastener releasably engaged with the hook fastener such that the straps can wrap around the body portion 94 of the user and define a space receiving the body portion 94 with the treatment part 92.

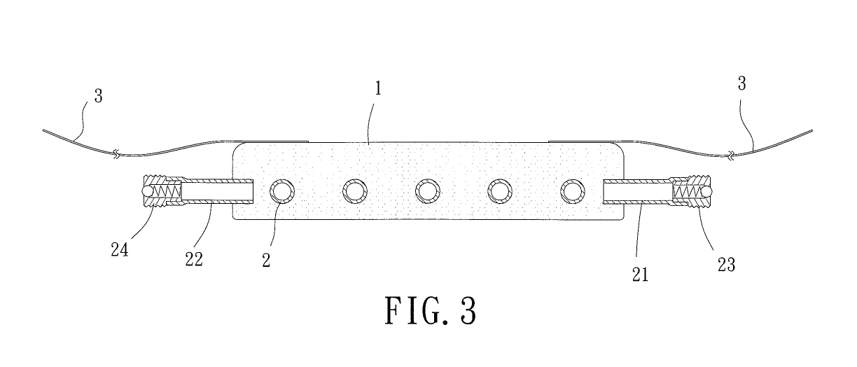

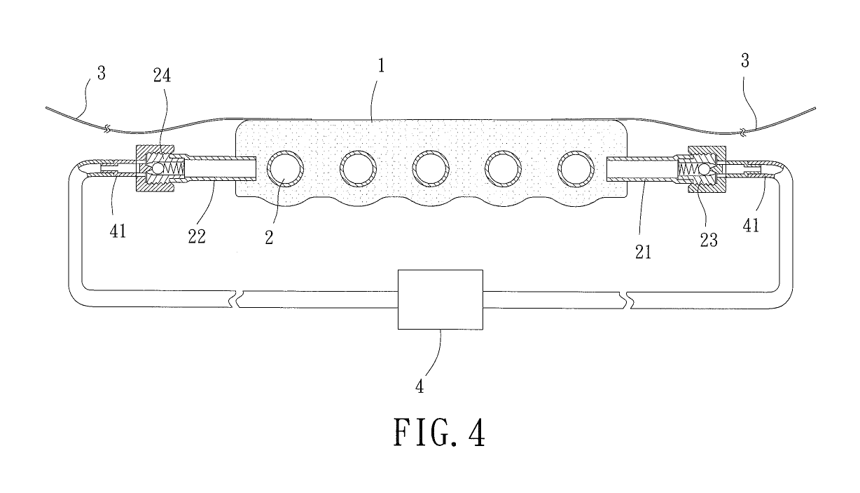

In addition, referring to FIGS. 3 and 4, a pressure device 4 is connected with the two check valves 23 and 24 respectively through a tube 41 which may be a pump. The shape of the container 2 will remain unexpanded when the pressure device 4 is not operated. The container 2 will expand and increase its thickness when the pressure device 4 fills the container 2 with the fluid via the tube 41, imparting a pressure to the treatment part 92 through the container 2.

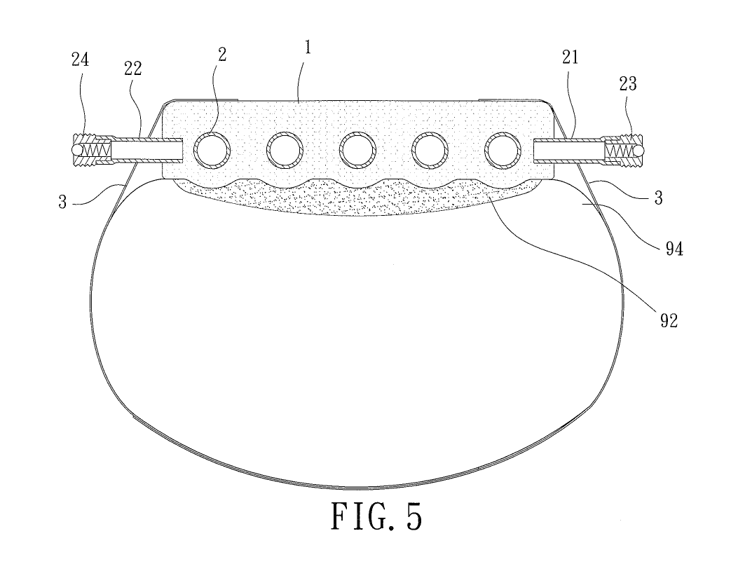

FIGS. 4 and 5 shows a use of the pressure bandage according to the preferred teachings of the present invention, with the bottom surface of the container 2 being in contact with the treatment part 92 of the user and the pressure bandage being fixed by the fixing member 3 to the body portion 94 with the treatment part 92.

The fluid is delivered into the container 2 via the tube 41 to expand the container 2 when the pressure device 4 is operated. Thus, the container 2 remains a positive pressure having a magnitude greater than the atmospheric pressure. Besides, the pressure device 4 can adjust and maintain the pressure in the container 2 within a desired range.

If the user is intended to move a distance beyond the length of the tube 41, the two check valves of the container 2 can be detached from the two ends of the tube 41 respectively after the pressure device 4 pressurized the container 2 to a positive pressure status. Therefore, the pressure bandage of the present invention can allow the free movement of the user. Since the two check valves are respectively used as the inlet and outlet of the container 2 for limiting the flow of the fluid, the container 2 allows continuous use of the pressure bandage under a pressurized status. The two check valves are reconnected with the tube 41 respectively in order to adjust the positive pressure applied to the treatment part 92 when the pressure difference between the atmospheric pressure and the pressure imparted by the pressure bandage to the treatment part 92 becomes small.

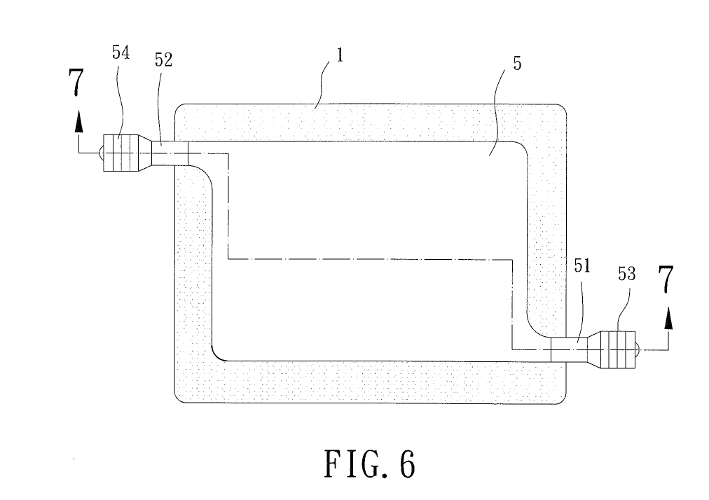

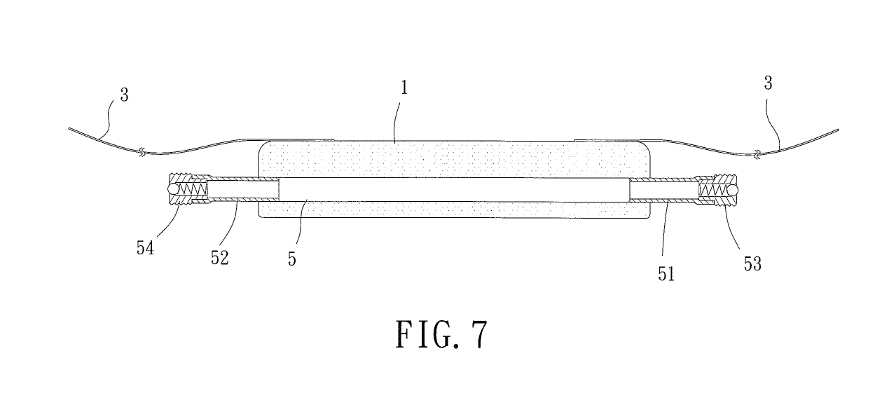

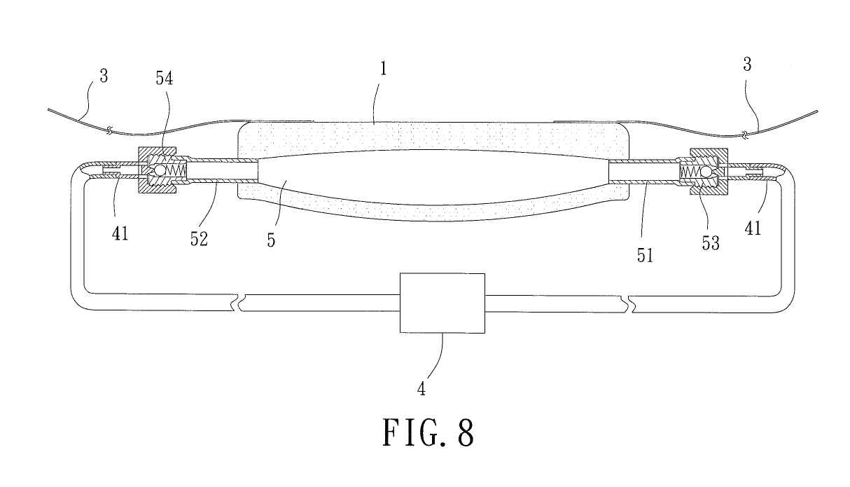

Referring to FIGS. 6, 7 and 8, a pressure bandage is disclosed according to a second embodiment of the present invention is shown. In comparison with the first embodiment, what is different from the previous embodiment is that the container 2 is in the form of a bladder made of polypropylene, polyethylene or rubber, etc. With delivering the fluid into the container 2 via the pressure device 4, the pressure bandage of present invention can continuously imparts the positive pressure to the treatment part 92 of the user.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A pressure bandage comprising:
a banding porting;
a container adapted to receive a fluid, with the container including a first and a second connecting portions respectively protruded from the outer periphery of the banding portion, with a check valve mounted in at least one of the first and second connecting portions for delivering the fluid into the container; and
at least one fixing member adapted to fix the banding portion to a treatment part.

2. The pressure bandage as claimed in claim 1, further comprising two check valves mounted in the first and second connecting valves, respectively.

3. The pressure bandage as claimed in claim 1, wherein the fixing member includes two straps respectively having a hook fastener and a loop fastener releasably engaged with the hook fastener such that the straps wrap around a body portion.

4. The pressure bandage as claimed in claim 1, wherein the container is fixed in the banding portion and adjacent to a contact surface of the banding portion.

5. The pressure bandage as claimed in claim 4, wherein the container is a conduit.

6. The pressure bandage as claimed in claim 1, wherein the container is a bladder.
